(19) United States
(12) Patent Application Publication
(10) Pub. No.: US 2008/0124391 A1
(43) Pub. Date: May 29, 2008

(54) ALGAL AND ALGAL EXTRACT DIETARY SUPPLEMENT COMPOSITION

(75) Inventors: David A. EVANS, Edmonton (CA); Melchior Rabie, Simondium (ZA)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: U.S. Nutraceuticals LLC dba Valensa International a Florida limited liability company, Eustis, FL (US)

(21) Appl. No.: 11/564,025

(22) Filed: Nov. 28, 2006

Related U.S. Application Data
(60) Provisional application No. 60/740,010, filed on Nov. 28, 2005.

(51) Int. Cl. A61K 9/64; A01N 63/00; A61K 9/48; A23L 1/30; A61K 9/50; A61K 36/02

(52) U.S. Cl. 424/456; 424/93.4; 424/195.17; 424/451; 424/502; 426/72

(57) ABSTRACT

A human dietary supplement composition comprising the dried biomass of blue-green algae (Spirulina platensis) and astaxanthin is disclosed, The composition is stable, effective and convenient for supporting the health and well-being of either healthy or health-challenged humans. Further, a method of ameliorating viral infections in human subjects by administering the composition to such subjects is disclosed.

### FIELD OF THE INVENTION

The present invention relates to dietary supplements, and, more particularly to such supplements and their method of manufacture based upon botanical materials.

### BACKGROUND OF THE INVENTION

The natural dietary supplement industry represents a $300 billion dollar marketplace worldwide. Many natural botanical materials and extracts have been used by mankind for health purposes for thousands of years. In some parts of the world, natural health products are preferred over chemical or pharmaceutical ones due to reasons of religion, culture, safety, cost and demonstrated efficacy.

Among the botanical products that have a history of use in support of human health are the algae. Two algae that are known to be used to support human health are Spirulina platensis and Haematococcus pluvialis.

Spirulina platensis.

Spirulina platensis Geitler is a mobile multicellular filamentous blue-green algae, which occurs naturally in the highly alkaline volcanic lakes of Africa and Mexico. It is now grown in cultured ponds in many countries of the world, including, Africa, India, China and the Hawaiian Islands of the USA.

Spirulina has been shown to enhance immune function and is specifically applicable to immune compromised people, such as those suffering from HIV/AIDS and malnutrition. This natural enhancement of immune function can be boosted further by raising the amount of the trace element selenium in the algae biomass. Selenium deficiency is commonly associated with HIV/AIDS (Patrick, 1999; Baum et al., 1997) Researchers believe that selenium may be important in HIV disease because of its role in the immune system and as an antioxidant.

A unique form of Spirulina is one produced in a closed controlled system protected from environmental contaminants where the algae culture media can be modified by the addition of chelated trace elements to “tailor” the natural organic composition of the biomass. In this way the trace element concentration in Spirulina can be enhanced. Selected trace elements are added as inorganic chelates at specific stages in the Spirulina growth cycle. These elements are then metabolized and converted into organic complexes within the organism prior to harvesting the algal biomass. For example, Selenium levels can be enhanced to a final concentration of at least 100 mg per kilogram of biomass dry matter.

Saeki et al. (2000) showed that both IFN-gamma secretion activity and NK cell damage activities were enhanced significantly after two weeks treatment with a 40% Spirulina hot water extract in over 40 year old males. Evets et al. (1994) disclose the use of 5 grams of Spirulina per day for 45 days as effective in normalizing above average IgE levels observed in children in highly radioactive areas of Russia.

In vitro studies (Quereshi et al., 1995a; Quereshi et al., 1995b) have shown that chicken macrophages treated with a water extract of Spirulina resulted in immune stimulation in the form of increased macrophage function, antibody response and phagocytosis. Complementary studies in which chickens were fed levels up to 1.6% Spirulina in the diet showed an approximately two-fold higher cutaneous basophilic hypersensitivity (CBH) response after injection with phyohemagglutinin-P (PHA-P) and elicited T-cell responses nearly four-fold greater than controls. Al-Batshan et al. (2001), again working with chickens, showed that Spirulina feeding upregulates macrophage phagocytic as well as metabolic pathways leading to increased nitric oxide activity. This is known to have a positive immunomodulatory effect since antimicrobial effects of nitric oxide, produced by macrophages, against pathogenic micro-organisms, including bacteria, viruses and protozoa, is well documented.

Hayashi et al. (1996) isolated from Spirulina platensis a novel sulphated-polysaccharide, calcium spirulan (Ca-SP), that inhibits the replication in vitro of several enveloped viruses including Herpes simplex type I, human cytomegalovirus, measles virus, mumps virus, influenza A virus and HIV-1 virus.

Feeding rats a diet with 5% spirulina for 100 days (compared to a control group not fed spirulina) revealed: 1. the weight of the caecum increased 13%; 2. lactobacillus increased 327%; 3. vitamin B₁ (thiamine) inside the caecum increased 43%. Since spirulina did not supply this additional B₁, it improved overall B₁ absorption. The study suggests eating spirulina increases lactobacillus and may increase efficient absorption of Vitamin B₁ and other vitamins from the entire diet (Tokai et al., 1997).

The blue-green algae, Spirulina platensis, has been used for hundreds of years as a food source for humans and animals due to the excellent nutritional profile and high carotenoid content. Spirulina is relatively high in protein with values ranging from 55-70% and includes all of the essential amino acids (Clement et al., 1967; Bourges et al., 1971; Anusuya Devi et al., 1981; Biodelta, 1994). The available energy has been determined to be 2.5-4.3 kcal/gram with a phosphorus availability of 41% (Yoshida and Hoshii, 1980′ Biodelta, 1994). Although Spirulina powder appears as a bluish-green color, in fact it contains one of the highest levels of carotenoids of any natural food source when properly cultivated and processed (Matsuno et al., 1974; Tanaka et al., 1974; Nells and De Leenheer, 1983; Miki et al., 1986). Carotenoids are a family of over 600 natural lipid-soluble pigments that are primarily produced within phytoplankton, algae and plants. Some fungal and bacterial species can also synthesize carotenoids, but animals cannot produce them de novo. Within the various classes of natural pigments, the carotenoids are the most widespread and structurally diverse pigmenting agents. Carotenoids are responsible for a wide variety of colors in nature, the most notable are the brilliant yellow to red colors of fruits and leaves of plants. In combination with proteins, carotenoids also contribute to the wide range of blue, green, purple, brown and reddish colors of fish, insect, bird and crustacean species. These natural pigments help protect cells against light damage, but the pigments have broader functions in various organisms as precursors to vitamin A, antioxidant activity in quenching oxygen radicals, immune enhancement, hormone regulation, and additional roles in growth, reproduction and maturation. The major carotenoids of Spirulina are β-carotene, β-cryptoxanthin and zeaxanthin.

Spirulina is traditionally used in dried powder biomass form and is traditionally taken orally at a daily rate of about 45 mg per kilogram of bodyweight. In the preferred form, the powder is taken as 500 mg to 1000 mg tablets or dispersed in beverage.

Astaxanthin

Astaxanthin (3,3′-dihydroxy-β,β-carotene-4,4′-dione) CAS [471-53-4], is a keto carotenoid pigment naturally accumulated via the diet in marine animals such as salmon, shrimp, red seabream and lobster and in birds such as flamingoes. Astaxanthin also occurs in certain microalgae such as Haematococcus pluvialis and in yeasts such as Phaffia species. The highest concentration, up to four percent of dry matter, occurs in Haematococcus. It can also be chemically synthesized, but not in only naturally occurring stereoisomer form.

Astaxanthin, although related to other carotenoids such as beta-carotene, zeaxanthin and lutein, is a more powerful antioxidant. Astaxanthin is particularly potent in quenching singlet oxygen and has over five hundred times the ability to quench singlet oxygen as alpha-tocopherol. This antioxidant activity of astaxanthin is thought to be responsible for the wide range of health-promoting properties it exhibits, including skin and eye protection from damage by UV-light, anti-inflammatory activity, modulation or promotion of the immune response, reduction in ageing processes and benefits to heart, liver, joints and prostate. An excellent review of astaxanthin's health promoting properties is given by Guerin et al. (2003).

Tso, et al. (1996) disclosed the use of astaxanthin as a method of retarding or ameliorating central nervous system and eye damage, especially age-related macular degeneration.

As with Spirulina, astaxanthin shows significant immune response modification, but in contrast, acts as a lipophilic agent.

Lorenz (2002) discloses the use of astaxanthin as an oral or topical treatment to retard, ameliorate and prevent canker sores

Lignell and Bottiger (2004), disclose the use of astaxanthin to suppress excessive Th1 cell mediated immune responses and stimulating Th2 cell mediated immune responses in human patients with Crohn's Disease. Although only Crohn's Disease was studied, these authors speculate that, “it is likely that patients suffering from other predominantly Th1 cell mediated diseases would benefit . . . ”, Unfortunately no measurements of Th1 or Th2 mediated responses were made and the immune mediating role postulated for astaxanthin in this disclosure is purely speculative.

Chew et al., (2004), disclose a composition comprising astaxanthin for use by a companion animal for attenuating inflammation, enhancing immunity, enhancing longevity, and combinations thereof. In this case the authors showed that astaxanthin was responsible for both cell-mediated and humoral immune responses in the subject dogs and cats.

Chew (2004) completed a randomized, placebo controlled, dose escalating, double blind human trial with astaxanthin which clearly demonstrated astaxanthin's role in stimulating human immune response in normal subjects,

More recent work (Chew and Park, 2005) discloses that astaxanthin also effectively reduces non-specific environmental damage to cellular DNA in normal human subjects, especially that of immune cells. In this study, astaxanthin was in the form of a supercritical carbon dioxide extract of H. pluvialis.

Most recently, Kotwal (2006) has shown antiviral activity with astaxanthin esters extracted from Haematococcus after encapsulation into a beadlet form.

Astaxanthin is consumed for health purposes at a daily rate of from about 0.01 mg per kilogram of bodyweight to about 0.20 mg per kilogram of bodyweight.

The following references are further illustrative of the background of the invention.

### U.S. PATENTS AND PUBLISHED APPLICATIONS

U.S. Pat. No. 5,527,533 Tso, et al. 6/1986
U.S. Pat. No. 6,344,214 B1 Lorenz, R. Todd. 2/2002
U.S. Pat. No. 6,733,708 B1 Lignell, et al. 8/2004
20040151761 Chew, Boon P. et al. 8/2004
2005011712 Chew, Boon P. et al. 2/2005

### TECHNICAL REFERENCES

Al-Batshan H. A., Al-Mufarrej S. I., Al-Homaidan A. A., Qureshi M. A. 2001. Enhancement of chicken macrophage phagocytic function and nitrite production by dietary Spirulina platensis. Immunopharmacol Immunotoxicol. 23(2):281-9.

Anusuya Devi M., Subbulakshimi G., Madhavi Devi K., Venkataram L. V. 1981. Studies on the proteins of mass-cultivated, blue-green alga (Spirulina platensis). J. Agric. Food Chem. 29: 522-525.

Baum, M. K., Shor-Posner G., Lai S., Zhang G., Lai H., Fletcher M. A., Sauberlich H., Page J. B. 1997. High risk of HIV-related mortality is associated with selenium deficiency. J Acquir Immune Defic Syndr Hum Retrovirol. 15(5):370-4.

Bourges H., Sotomayor A., Mendoza E., Chavez A. 1971. Utilization of the algae Spirulina as a protein source. Nutr. Rep. Int. 4:31-43.

Clement G., Giddey C., Menzi R. 1967. Amino acid composition and nutritive value of the algae Spirulina maxima. J. Sci. Food Agric. 18:497-501.

Evets L. B., Belookaya T., Lyalikov S., Orehov S. D., Shipulin E. 1994. Means to normalize the levels of immunoglobulin E. Russian Federation Committee of Patents and Trade. Patent Number (19)RU (11)20005486 C1 {51} 5 A 61K35/80. 1-page translation.

Guerin, M. Huntley, M. E., Olaizola, M. 2003. Haematococcus astaxanthin: applications for human health and nutrition. Trends Biotech. 21:5 210.

Hayashi T. and Hayashi K. 1996. Calcium Spirulan, an inhibitor of enveloped virus replication, from blue-green alga Spirulina platensis. J. Nat. Prod. 59:83-87.

Kotwal, G. J., Hugin, A. W., Moss, B. 1989. Mapping and instertional mutagenesis of vaccinia virus gene encoding a 13,800-Da secreted protein. Virology. 171(2):579-87

Kotwal, Girish J. 2005. Confidential Communication, Jun. 27, 2006.

Matsuno T., Nagata S., Iwahashi M., Koike T., Okada M. 1974. Intensification of color of fancy red carp with zeaxanthin and myxoxanthophyl, major carotenoid constituents of Spirulina. Bul. Jpn. Soc. Sci. Fish. 45: 627-632.

Miki W., Yamaguchi K., Konosu S. 1986. Carotenoid composition of Spirulina maxima. Bull. Jpn. Sco. Sci. Fish. 52(7): 1225-1227.

Nells H. J. C. F. and De Leenheer A. P. 1983. Isocratic nonaqueous reversed-phase liquid chromatography of carotenoids. Anal. Chem. 55: 27-275.

Qureshi M. A., Kidd M. T., Ali R. A. 1995a. Spirulina platensis extract enhances chicken macrophage functions after in vitro exposure. Journal of Nutritional Immunology. 3(4): 35-45.

Qureshi M. A., Ali R. A., Hunter R. 1995b. Immunomodulatory effects of Spirulina platensis supplementation in chickens. Proc. 44^{th} Western Poultry Disease Conference, Sacramento, Calif. 117-121.

Saeki Y., Matsumoto M., Hayashi A., Azuma I., Toyoshima K., Seya T. 2000. The effect of Spirulina hot water extract to the basic immune activation. Summary of paper presented at the 30^{th} Annual Meeting of the Japanese Society for Immunology. Nov. 14-16, 2000.

Tanaka Y., Matsuguchi H., Katayama T. 1974. Comparative biochemistry of carotenoids in algae-IV: Carotenoids in Spirulina platensis. Mem. Fac. Fisch. Kagoshima Univ. 23: 111-115.

Tokai Y., et al. 1987. Effects of spirulina on caecum content in rats. Chiba Hygiene College Bulletin. February 1987 Vol. 5, No. 2. Japan.

Yoshida M. and Hoshii H. 1980. Nutritive value of Spirulina, green algae, for poultry feed. Japan Poultry Sci. 17: 27-30.

### SUMMARY OF THE INVENTION

It is clear from the wide ranging health-promoting properties of both Spirulina platensis and astaxanthin that there are a number of areas where the two are complementary in action and a number of areas where they supplement each other. For example, both products appear to modulate the immune system, Spirulina through largely water-soluble components and astaxanthin through fat soluble components. Alternatively, astaxanthin is known to provide antioxidant protection at the cellular level but Spirulina has not been shown to have this property. Further, Spirulina is known to exhibit anti-viral activity through lipophobic components whereas astaxanthin is disclosed herein to exhibit novel anti-viral activity through a lipophilic component.

It is clearly desirable to formulate a product combining the two supplements to create an improved dietary supplement, with broader health-promoting properties. That is, a product composition combining lipophobic and lipophilic components each with immune and anti-viral properties, simultaneously capable of providing anti-viral activity, immune system support and cellular protection. Such a composition would be of immense value to both healthy and health-challenged humans especially those infected with HIV.

The preferred form of dried Spirulina biomass to use in the composition is from Spirulina produced in a closed system and dried at a low temperature to minimize damage to sensitive components. It is also desirable to increase the organic-bound selenium level of the dried biomass by adding selenium to the culture media while growing the Spirulina. In certain countries where selenium-supplemented Spirulina is prohibited from sale by law, the selenium can be added directly during formulation.

Astaxanthin used in the formulation can be derived from natural astaxanthin-containing sources, such as for example, Haematococcus algae, Phaffia and other microorganisms, or from synthetic manufacture. The synthetic form is less preferred as it is not approved for human use (FDA, USA) , and does not consist entirely of natural isomers.

The astaxanthin can be used in the form of astaxanthin-bearing dried biomass and/or astaxanthin-bearing extracts, of Haematococcus, Phaffia or other natural organisms. Unfortunately, when whole dried Haematococcus or Phaffia biomass is ground to a powder the astaxanthin rapidly loses potency through exposure to a combination of air, lipases and pro- oxidants in the organism itself. Such ground biomass powders should be stored in vacuum-packed oxygen impermeable bags or frozen, to prevent loss of astaxanthin. Consequently the composition comprising a mixture of dried biomasses is less preferable.

The preferred form of astaxanthin to use in the composition is an extract derived from Haematococcus pluvialis Flotow. H. pluvialis can be cultured in closed systems and produced free of environmental contamination which might occur in open-pond systems. H. pluvialis produces up to four percent by weight of dry matter as astaxanthin, making it the most economical source for natural astaxanthin. The stereoisomers of astaxanthin produced by H. pluvialis are identical to those occurring naturally in salmon and hence, in the human diet. Additionally, H. pluvialis produces the astaxanthin largely (over 90%) in esterified form which is much more stable than in the free form.

When H. pluvialis is dried, ground and extracted with supercritical carbon dioxide, the astaxanthin esters are obtained as an oily, viscous dark red extract which, under appropriate storage conditions, is stable for greater than two years. Consequently this extract is the preferred form of astaxanthin for inclusion in the composition. To increase stability further, particularly in a tablet delivery system, the astaxanthin-bearing extract of H. pluvialis can be encapsulated in maltodextrin, gelatin, etc., by well known processes such as, for example, spray drying.

The composition disclosed here (SpiruZan™), comprises the mixing of dried powder biomass of Spirulina platensis with a suitable form of astaxanthin. The astaxanthin is preferably encapsulated in a suitable carrier matrix to enhance stability. The thoroughly mixed composition is then converted to a tablet in a standard tableting machine. The composition is designed to contain from about 0.025 percent astaxanthin to about 2.5 percent astaxanthin by weight. The preferred tablet weight is 500 mg so that the preferred daily intake is two tablets taken 3 times per day and supplying about 3 grams of dried Spirulina biomass together with 4 to 5 mg of natural astaxanthin.

### DETAILED DESCRIPTION OF THE PREFERRED EMBODIMENTS

According to one aspect of the invention a dietary supplement composition is disclosed that combines the dried biomass of blue-green algae (Spirulina platensis) with a suitable form of astaxanthin. In a preferred form of the invention, the astaxanthin is in the form of an astaxanthin-bearing extract of Haematococcus pluvialis. The composition discloses a range for addition of the astaxanthin-bearing extract to the dried Spirulina biomass from about 0.025 percent astaxanthin to about 2.5 percent astaxanthin by weight.

According to another aspect of the invention the astaxanthin may be preferably encapsulated in an encapsulating agent prior to incorporation in the composition in order to minimize oxidative deterioration of the astaxanthin when in contact with the dried Spirulina biomass. Typical encapsulating agents include for example, starches, maltodextrins, gelatins, proteins, polymers, sugars and polysaccharides. Such a disclosed dietary supplement composition can be conveniently processed into tablets, capsules and powders that exhibit excellent oxidative stability and facilitate oral administration. When ingested by humans or animals, the one composition supports health and well-being associated with the two separate algae in an effective and convenient single-dose concept. Normal daily intake is in the preferred range of about 45 mg of composition per kilogram of bodyweight. For the average adult human this is equivalent to about 3 grams of composition per day, conveniently taken as two 500 mg doses, three times per day. Those skilled in the art will be aware that 500 mg is a convenient unit dose for tablets and capsules.

It will be understood by those in the art that such a composition, in tablet, capsule or powder may be conveniently supplemented with other biologically active extracts and compounds, including for example: vitamins, minerals, antioxidants, tocopherols, tocotrienols, phytosterols, fatty alcohols, polysaccharides and bioflavonoids.

According to another aspect of the invention, the selenium content of the Spirulina platensis used in the composition can be increased naturally by feeding the growing algae a selenium rich substrate prior to harvesting and drying. Selenium contents of about 100 mg per kilogram of dried biomass are conveniently achieved in this way. In certain countries where selenium-supplemented Spirulina is prohibited from sale by law, the selenium can be added directly during formulation.

A higher selenium intake is known to be desirable in some health-challenged humans and animals and also in selenium deficit areas of the world where a sufficient daily intake of selenium might not be received through the local diet.

According to another aspect of the invention it is disclosed that a daily intake of the composition of from about 10 mg per kilogram of body weight to about 150 mg per kilogram of body weight effectively supports the health and well-being of both healthy and health-challenged humans.

The following examples are illustrative of the present invention, and are not to be construed as limiting thereof.

### EXAMPLE 1

Spirulina platensis was cultivated in a closed production system supplemented with selenium. The mature algae was harvested, dried and ground into a fine powder. The selenium content was 100 mg per kilogram of dried biomass.

TABLE 1
TYPICAL ANALYSIS OF DRIED SELENIUM-ENHANCED BIOMASS
(Average value per kg)
Common value

| Protein | 68% | |
|---|---|---|
| Energy | 19.18 | MJ |
| Carbohydrates | 150 | g |
| Lipids | 55 | g |
| Minerals | 120 | g |
| Moisture | 70 | g |

Pigments and Enzymes

| | | |
|---|---|---|
| Beta-carotene | 1.26 | g |
| Total carotenoids | 1.75 | g |
| Xanthophylls | 1.18 | g |
| Chlorophyll-a | 10.15 | g |
| Phycocyanin | 128 | g |

Vitamins

| | | |
|---|---|---|
| Thiamin (B1) | 26 | mg |
| Riboflavin (B2) | 34 | mg |
| Niacin (B3) | 148 | mg |
| Pantothenic acid (B5) | 6 | mg |
| Pyridoxine (B6) | 4 | mg |
| Cyanocobalamin (B12) | 0.7 | mg |
| α, δ tocopherol (E) | 109 | mg |
| Biotin (H) | 0.3 | mg |
| Folic acid | 0.6 | mg |
| Inositol | 1100 | mg |

Essential Amino Acids

| | | |
|---|---|---|
| Isoleucine | 39 | g |
| Leucine | 58.6 | g |
| Lysine | 33.5 | g |
| Methionine | 14.2 | g |
| Phenylalanine | 26.1 | g |
| Threonine | 33 | g |
| Tryptophan | 10.4 | g |
| Valine | 30.6 | g |

Non-essential Amino Acids

| | | |
|---|---|---|
| Alanine | 53.4 | g |
| Arginine | 41.9 | g |
| Aspartic acid | 84.2 | g |
| Cysteine | 6.1 | g |
| Glutamic acid | 85.7 | g |
| Glycine | 33.8 | g |
| Histidine | 10.3 | g |
| Proline | 29.1 | g |
| Serine | 33.1 | g |
| Tyrosine | 31.4 | g |

Minerals & Trace Elements

| | | |
|---|---|---|
| Magnesium | 8040 | mg |
| Calcium | 5370 | mg |
| Phosphorus | 10100 | mg |
| Potassium | 19500 | mg |
| Sodium | 12600 | mg |
| Chloride | 1080 | mg |
| Iron | 986 | mg |
| Selenium | 100 | mg |
| Cobalt | 15 | mg |
| Chromium | 2 | mg |
| Arsenic | <1 | mg |
| Lead | 1 | mg |
| Mercury | <1 | mg |
| Cadmium | 0.2 | mg |
| Nickel | 11 | mg |
| Manganese | 71 | mg |
| Molybdenum | 5.9 | mg |
| Copper | 9.5 | mg |
| Zinc | 59 | mg |
| Barium | 0.8 | mg |
| Boron | 21 | mg |
| Iodine | 4 | mg |
| Titanium | 13.5 | mg |
| Vanadium | 3 | mg |

Fatty Acids

| | | |
|---|---|---|
| γ-Linolenic acid (GLA) | 8160 | mg |
| Linoleic acid | 9760 | mg |
| Oleic acid | 1970 | mg |
| Palmitic acid | 16900 | mg |
| Palmitoleic acid | 1540 | mg |
| Stearic acid | 480 | mg |

Microbial

| Total plate count | <10⁶ | CFU/g |
|---|---|---|
| E. coli | none | |
| Salmonella | none | |
| Staphylococcus | none | |
| Yeast & Moulds | <100 | CFU/g |

### EXAMPLE 2

Haematococcus pluvialis was cultivated in a closed system, harvested, de-watered, cracked to break the cell walls and dried. This dry biomass was extracted with supercritical carbon dioxide at a pressure of 650 bar and a temperature of 60 degrees Celsius. A dark red extract was obtained containing 10% astaxanthin with the typical composition shown in Table 2.

TABLE 2
TYPICAL ANALYSIS OF
ASTAXANTHIN-RICH H. pluvialis EXTRACT
(Average value per kg)
Common value

| Protein | <1% | |
|---|---|---|
| Energy | 36.7 | MJ |
| Carbohydrates | <1 | g |
| Lipids | 971 | g |
| Minerals | 24 | mg |
| Moisture | 2 | g |

Carotenoids

| | | |
|---|---|---|
| Astaxanthin | 100 | g |
| Beta-carotene | 0.3 | g |
| Lutein | 0.7 | g |
| Canthaxanthin | 0.3 | g |
| Total carotenoids | 102 | g |

Vitamins

| | | |
|---|---|---|
| Tot. mixed tocopherols (E) | 100 | mg |

Minerals & Trace Elements

| | | |
|---|---|---|
| Calcium | 12 | mg |
| Phosphorus | 4.6 | mg |
| Sodium | 1.3 | mg |
| Iron | 1.2 | mg |
| Selenium | 1.1 | mg |
| Arsenic | <1 | mg |
| Lead | <1 | mg |
| Mercury | <1 | mg |
| Zinc | 1.2 | mg |
| Silicon | 1.2 | mg |
| Tin | 1.2 | mg |

Fatty Acids

| | | |
|---|---|---|
| Palmitic acid | 83 | g |
| Hexadecatrienoic | 16 | g |
| Hexadecatetraenoic | 43 | g |
| Oleic acid | 179 | g |
| Linoleic acid | 241 | g |
| α-Linolenic acid | 15 | g |
| γ-linolenic acid | 102 | g |
| Stearidonic acid | 15 | g |
| Eicosatetraenoic acid | 9 | g |
| Eicosapentaenoic acid | 46 | g |

Microbial

| Total plate count | <10³ | CFU/g |
|---|---|---|
| E. coli | none | |
| Salmonella | none | |
| Staphylococcus | none | |
| Yeast & Moulds | <100 | CFU/g |

It can be seen from Table 2 that the astaxanthin-rich H. pluvialis extract contained 10.2% Total Carotenoids of which 98% is in the form of astaxanthin, being 10 grams per kilogram of extract. The extract also contains over 5% of eicosapentaenoic acid (EPA) and eicosatetraenoic acid (ETA) in total and over 10% of the polyunsaturated fatty acids, linoleic (18:2)and linolenic (18:3).

### EXAMPLE 3

The astaxanthin-bearing extract was emulsified in a gelatin substrate and spray dried to produce an encapsulated fine beadlet product, with the typical composition shown in Table 3.

TABLE 3
TYPICAL ANALYSIS OF ASTAXANTHIN EXTRACT BEADLET
(Average value per kg)
Common value

| Protein | 28.5% | |
|---|---|---|
| Energy | 22.9 | MJ |
| Carbohydrates | 315 | g |
| Lipids | 335 | g |
| Moisture | 48 | g |

Carotenoids

| | | |
|---|---|---|
| Astaxanthin | 26 | g |
| Beta-carotene | 0.08 | g |
| Lutein | 0.18 | g |
| Canthaxanthin | 0.08 | g |
| Total carotenoids | 26.3 | g |

Vitamins

| | | |
|---|---|---|
| Mixed tocopherols (E) | 109 | mg |

Essential Amino Acids

| | | |
|---|---|---|
| Leucine | 10 | g |
| Lysine | 13 | g |
| Phenylalanine | 6 | g |
| Threonine | 6 | g |

Non-essential Amino Acids

| | | |
|---|---|---|
| Alanine | 34 | g |
| Arginine | 27 | g |
| Glutamine | 35 | g |
| Glycine | 81 | g |
| Proline | 48 | g |
| Hydroxyproline | 42 | g |
| Serine | 11 | g |

Minerals & Trace Elements

| | | |
|---|---|---|
| Arsenic | <1 | mg |
| Lead | <1 | mg |
| Mercury | <1 | mg |

Fatty Acids

| | | |
|---|---|---|
| Palmitic acid | 44.5 | g |
| Hexadecatrienoic | 4.2 | g |
| Hexadecatetraenoic | 11.2 | g |
| Oleic acid | 46.5 | g |
| Linoleic acid | 62.7 | g |
| α-Linolenic acid | 3.9 | g |
| γ-Linolenic acid | 26.5 | g |
| Stearidonic acid | 3.9 | g |
| Eicosatetraenoic acid | 2.3 | g |
| Eicosapentaenoic acid | 12 | g |

Microbial

| Total plate count | <10³ | CFU/g |
|---|---|---|
| E. coli | none | |
| Salmonella | none | |
| Staphylococcus | none | |
| Yeast & Moulds | <100 | CFU/g |

It can be seen from Table 3 that the astaxanthin extract beadlets contained about 2.5% by weight of astaxanthin. The beadlets are of a fine granular form very suitable for delivering astaxanthin to a tableting process. Such beadlets can be conveniently mixed with Spirulina platensis powder to be formed into tablets or capsules without exposing the astaxanthin to oxidative processes.

### EXAMPLE 4

A sample of the beadlets described in Example 3 was subjected to an in vitro screen for both antiviral and toxic activity against the poxvirus vGK5 (Kotwal et al, 1989).

The poxvirus stock of known amount of virus in 100 μL was mixed with 5, 10 or 20 μL of the diluted (1:10 and 1:4) beadlet material [treatment bd-1] for approximately 1 minute. It was then added to 1 mL of medium covering a well of a 6 well plate. After mixing, 100 μL was transferred to the next well and so on until a dilution of 10⁻⁶ or 10⁻⁷ was obtained. The plate was then incubated for 48 hours after which the medium was removed and stained with 0.1% crystal violet solution. A control plate without the addition of beadlet material was also done [control bd-2] to get the exact count of the virus without treatment. The percent inhibition was then determined as the ratio of actual count after treatment divided by the total count without treatment multiplied by 100.

The beadlets were tested as is and the inert excipients (all ingredients excluding astaxanthin extract) tested separately. Antipoxviral inhibition was tested at concentrations of 3.0×10⁶ PFU/mL, 2.4×10⁶ PFU/mL and 2.7×10⁶ PFU/mL. At 5 μl, 10 μl and 20 μl a 1:10 dilution of the beadlets showed inhibitions in the range of 85% to 100%. Linear regression adjustment to a poxvirus concentration of 1×10⁶ PFU/mL yielded estimated inhibitions of 107% (5 μL), 94% (10 μL) and 72% (20 μL). Equivalent regression inhibition estimates for the excipients were 0% (6 μL), 0% (12 μL) and 5% (24 μL). The excipients showed no significant inhibition (0% to 62%) over 6, 12 and 24 μL/mL.

The 1:10 beadlet dilution showed zero toxicity at the 5 μL level, but maintained high toxicity at the 10 μL and 20 μL doses. The excipients showed no toxicity at the 6, 12 and 24 μL doses, even when diluted 1:4.

It is clear that the beadlet material showed 100% inhibition of the poxvirus vGK5 with zero toxicity when tested at the 5 μl dose of a 1:10 dilution. As the beadlets contain 2.5% astaxanthin, a 5 μl dose of a 1:10 beadlet dilution represents a 0.0125 μl dose of astaxanthin in 100 μl of poxviral solution or an effective concentration of 125 ppm. It is evident that the IC50 for astaxanthin in this study is significantly less than 125 ppm while the TC50 is significantly greater than 125 ppm.

TABLE 4

| | | | Inhibition at: | | | | |
|---|---|---|---|---|---|---|---|
| | | | 3.0 × 10⁶ | 2.4 × 10⁶ | 2.7 × 10⁶ | Calc. Inhibition at 1 × 10⁶ pfu/mL | |
| | | | pfu/mL | pfu/mL | pfu/mL | | trendline eq. |
| BD-1 | 5 | μL | 97.8 | 100 | 93.7 | 100 | y = 3.67x + 107 |
| | 10 | μL | 98.9 | 97.2 | 100 | 93.9 | y = 2.83x + 91 |
| | 20 | μL | 100 | 91.7 | 93.7 | 71.6 | y = 13.8x + 53 |
| BD-2 | 6 | μL | 27.8 | 0 | 62.3 | 0 | y = 46.3x − 95 |
| | 12 | μL | 52.2 | 0 | 62.3 | 0 | y = 87x − 196 |
| | 24 | μL | 26.7 | 22.2 | 5.7 | 5 | y = 7.5x − 2 |

### EXAMPLE 5

The encapsulated extract beadlets were blended in a dry mixer with the dried selenium-enhanced biomass at a level of 6.67% total weight. The resulting mixture was passed through a tableting machine, without the addition of binders, to form 500 mg tablets.

TABLE 5
TYPICAL ANALYSIS OF ASTAXANTHIN/
SELENIUM-ENRICHED SPIRULINA TABLETS
(Average value per kg)
Common value

| Protein | 65% | |
|---|---|---|
| Energy | 19.4 | MJ |
| Carbohydrates | 161 | g |
| Lipids | 73 | g |
| Minerals | 112 | g |
| Moisture | 68 | g |

Pigments and Enzymes

| | | |
|---|---|---|
| Astaxanthin | 1.67 | g |
| Beta-carotene | 1.18 | g |
| Xanthophylls | 1.10 | g |
| Total carotenoids | 3.95 | g |
| Chlorophyll-a | 9.47 | g |
| Phycocyanin | 119 | g |

Vitamins

| | | |
|---|---|---|
| Thiamin (B1) | 24.2 | mg |
| Riboflavin (B2) | 31.7 | mg |
| Niacin (B3) | 138 | mg |
| Pantothenic acid (B5) | 5.6 | mg |
| Pyridoxine (B6) | 3.7 | mg |
| Cyanocobalamin (B12) | 0.6 | mg |
| α, δ tocopherol (E) | 102 | mg |
| Biotin (H) | 0.3 | mg |
| Folic acid | 0.6 | mg |
| Inositol | 1030 | mg |

Essential Amino Acids

| | | |
|---|---|---|
| Isoleucine | 39 | g |
| Leucine | 58.6 | g |
| Lysine | 33.5 | g |
| Methionine | 14.2 | g |
| Phenylalanine | 26.1 | g |
| Threonine | 33 | g |
| Tryptophan | 10.4 | g |
| Valine | 30.6 | g |

Non-essential Amino Acids

| | | |
|---|---|---|
| Alanine | 53.4 | g |
| Arginine | 41.9 | g |
| Aspartic acid | 84.2 | g |
| Cysteine | 6.1 | g |
| Glutamic acid | 85.7 | g |
| Glycine | 33.8 | g |
| Histidine | 10.3 | g |
| Proline | 29.1 | g |
| Serine | 33.1 | g |
| Tyrosine | 31.4 | g |

Minerals & Trace Elements

| | | |
|---|---|---|
| Magnesium | 7500 | mg |
| Calcium | 5010 | mg |
| Phosphorus | 9420 | mg |
| Potassium | 18200 | mg |
| Sodium | 11800 | mg |
| Chloride | 1010 | mg |
| Iron | 920 | mg |
| Selenium | 93 | mg |
| Cobalt | 14 | mg |
| Chromium | 1.8 | mg |
| Arsenic | <1 | mg |
| Lead | <1 | mg |
| Mercury | <1 | mg |
| Cadmium | 0.2 | mg |
| Nickel | 10 | mg |
| Manganese | 66 | mg |
| Molybdenum | 5.5 | mg |
| Copper | 8.9 | mg |
| Zinc | 55 | mg |
| Barium | 0.7 | mg |
| Boron | 20 | mg |
| Iodine | 3.7 | mg |
| Titanium | 12.6 | mg |
| Vanadium | 2.8 | mg |

Fatty Acids

| | | |
|---|---|---|
| Stearic acid | 448 | mg |
| Palmitic acid | 18700 | mg |
| Palmitoleic acid | 1440 | mg |
| Hexadecatrienoic | 280 | mg |
| Hexadecatetraenoic | 747 | mg |
| Oleic acid | 4940 | mg |
| Linoleic acid | 13300 | mg |
| α-Linolenic acid | 260 | mg |
| γ-linolenic acid | 9380 | mg |
| Stearidonic acid | 260 | mg |
| Eicosatetraenoic acid | 153 | mg |
| Eicosapentaenoic acid | 800 | mg |

Microbial

| Total plate count | <10⁶ | CFU/g |
|---|---|---|
| E. coli | none | |
| Salmonella | none | |
| Staphylococcus | none | |
| Yeast & Moulds | <100 | CFU/g |

It can be seen from Table 5 that the astaxanthin is included in the tablets at 0.167% by weight. The selenium content is 93 mg per kg. The preferred daily intake of 6 tablets, being 3 grams of formulation, supplies 5 grams of astaxanthin and 0.279 mg of selenium. The tablets did not crumble but showed excellent dissolution properties when mixed with water. They were in a form very suitable for oral ingestion by humans.

### EXAMPLE 6

Tablets prepared as in Example 5 were utilized in a human subject demonstration of utility. Under the direction of a natural products AIDS Clinic, a number of patients with Human Immunodeficiency Virus (HIV) volunteered to undergo a period of consumption of the tablets at a daily intake of 6 tablets per day. A protocol, informed consent and safety factors were independently evaluated and approved by Independent Review Consulting, Inc., an accredited Institutional Review Board prior to the initiation of the intake period. 33 research subjects (26 men and 6 women) volunteered to participate in the demonstration. Three volunteers withdrew from the study; two for reasons unrelated to the supplement and one due to a possible allergic reaction. A further 17 subjects have expressed an interest in joining the demonstration. Subsequent overall health status of all participants will be evaluated.

It is clear from Example 6. that there is a significant need for the disclosed composition among the HIV and AIDS challenged population. It is also evident that the disclosed composition is convenient to administer and ingest without untoward difficulties. The present invention may be considered as a human dietary supplement composition comprising the dried biomass of Spirulina platensis in combination with astaxanthin. The astaxanthin is preferably in the form of an astaxanthin-bearing extract of Haematococcus pluvialis. The extract may be produced by supercritical fluid extraction with carbon dioxide, for example. The compositions may include the astaxanthin in a range of from about 0.025 percent (0.025%) by weight of the Spirulina platensis dried biomass to about 2.500 percent (2.500%) by weight of the Spirulina platensis dried biomass. The dried biomass of Spirulina platensis may have a selenium content increased by selenium supplementation of the feed substrate during the algal growing phase. For example, the selenium content of the Spirulina platensis dried biomass may be from about 50 mg per kilogram to about 150 mg per kilogram. In certain countries where selenium-supplemented Spirulina is prohibited from sale by law, the selenium can be added directly during formulation.

The astaxanthin may be micro-encapsulated in encapsulating agents such as including starches, maltodextrins, gelatin, polymers, proteins, polysaccharides and sugars. Other biologically active extracts and compounds may be added to the composition, such as, for example, vitamins, minerals, antioxidants, tocopherols, tocotrienols, phytosterols, fatty alcohols, polysaccharides and bioflavonoids. The composition may be provided in the form of tablets, capsules and powders, and may be incorporated in foods, feeds and beverages.

The composition may be used as dietary supplements to support the health and well-being of either healthy or health-challenged, humans. The composition may be used to ameliorate viral infections such as for example HIV/AIDS in human subjects. In particular, the composition may be administered orally to a human at a daily rate of from about 10 mg of composition per kilogram of body weight to about 150 mg of composition per kilogram of body weight and more preferably in the range of about 45 mg of composition per kilogram of body weight.

1. A human dietary supplement composition comprising the dried biomass of Spirulina platensis in combination with astaxanthin in free and/or ester form.

2. The composition of claims 1, in which the astaxanthin is derived from synthetic manufacture.

3. The composition of claim 1, in which the astaxanthin is contained in the astaxanthin-bearing dried biomass of Haematococcus, Phaffia or other natural organism.

4. The composition of claim 1, in which the astaxanthin is contained in an astaxanthin-bearing extract of Haematococcus, Phaffia or other natural organism.

5. The composition of claim 4, in which the astaxanthin-bearing extract is produced by supercritical fluid extraction with carbon dioxide.

6. The composition of claim 1, in which the astaxanthin and/or the astaxanthin-bearing dried biomass and/or the astaxanthin-bearing extract is micro-encapsulated in encapsulating agents including starches, maltodextrins, gelatin, polymers, proteins, polysaccharides and sugars.

7. The composition of claim 1, in which the astaxanthin typically comprises from about 0.025 percent (0.025%) by weight of the Spirulina platensis dried biomass to about 2.500 percent (2.500%) by weight of the Spirulina platensis dried biomass.

8. The composition of claim 1, in which the dried biomass of Spirulina platensis has the selenium content increased by selenium supplementation of the feed substrate during the algal growing phase.

9. The composition of claim 1, in which the selenium content of the composition is increased by the direct addition of selenium.

10. The composition of claim 8, in which the selenium content of the composition is from about 50 mg per kilogram of composition to about 150 mg per kilogram of composition.

11. The composition of claim 1, to which has been added other biologically active extracts and compounds, including for example, vitamins, minerals, antioxidants, tocopherols, tocotrienols, phytosterols, fatty alcohols, polysaccharides and bioflavonoids.

12. The composition of claim 1, in the form of tablets, capsules, powders, emulsions and gels.

13. The composition of claim 1, incorporated into foods, and beverages.

14. A method of supporting the health and well-being of either healthy or health-challenged human subjects by administering to the subject, the composition of claim 1.

15. A method of ameliorating the effect of viral infections in human subjects by orally administering to the subject the composition of claim 1.

16. A method of enhancing the health and well-being of human subjects inflicted with HIV and AIDS by orally administering to the subject the composition of claim 1.

17. The method of claim 14 where the composition is administered orally to a subject at a daily rate of from about 10 mg of composition per kilogram of body weight to about 150 mg of composition per kilogram of body weight and preferentially in the range of about 45 mg of composition per kilogram of body weight.

18. The method of claim 15, where the composition is administered orally to a subject at a daily rate of from about 10 mg of composition per kilogram of body weight to about 150 mg of composition per kilogram of body weight and preferentially in the range of about 45 mg of composition per kilogram of body weight.

19. The method of claim 16, where the composition is administered orally to a subject at a daily rate of from about 10 mg of composition per kilogram of body weight to about 150 mg of composition per kilogram of body weight and preferentially in the range of about 45 mg of composition per kilogram of body weight.
